(19) United States
(12) Patent Application Publication
(10) Pub. No.: US 2024/0108606 A1
(43) Pub. Date: Apr. 4, 2024

(54) A Rapamycin Composition

(71) Applicant: AFT Pharmaceuticals Limited, Auckland (NZ)

(72) Inventor: Hartley Atkinson, Auckland (NZ)

(21) Appl. No.: 18/038,409

(22) PCT Filed: Nov. 4, 2021
(86) PCT No.: PCT/NZ2021/050194

§ 371 Date: May 23, 2023

(30) Foreign Application Priority Data
Nov. 24, 2020 (AU) 2020277132

(51) Int. Cl. A61K 31/436; A61K 9/00; A61K 47/02; A61K 47/10; A61K 47/12; A61K 47/14; A61K 47/18

(52) U.S. Cl. CPC A61K 31/436; A61K 9/0014; A61K 47/02; A61K 47/10; A61K 47/12; A61K 47/14; A61K 47/183

(57) ABSTRACT

Rapamycin is a known medicinally therapeutic substance. A problem with Rapamycin is that it is quite unstable and prone to chemical degradation during storage. This can lead to medication becoming ‘under strength’ during its shelf life. It is an object of the invention to go at least some way towards addressing this. The invention is a composition for topical treatment, comprising rapamycin as active ingredient; vehicle comprising monolaurin, for example as glyceryl monolaurate and monomyristin, for example as glyceryl monomyristate; and water as a solvent.

### TECHNICAL FIELD

This invention relates to a rapamycin composition for administration to humans.

### BACKGROUND

Rapamycin was isolated in or around 1972 from samples of Streptomyces hygroscopicus. The compound was initially developed as an antifungal agent and its production is described in U.S. Pat. No. 3,929,992 to Ayerst McKenna & Harrison. Rapamycin has the following structural formula:

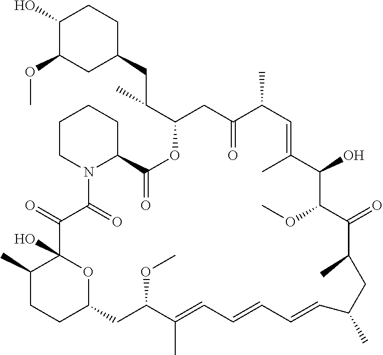 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

A problem with Rapamycin is that it is quite unstable and prone to chemical degradation during storage. This can lead to medication becoming ‘under strength’ during its shelf life.

### OBJECT OF THE INVENTION

It is an object of a preferred embodiment of the invention to go at least some way towards addressing the above problem. While this applies to the preferred embodiment, it should be understood that the object of the invention per se not so limited. It is simply to provide the public with a useful choice. Therefore any objects, advantages or benefits of any preferred embodiment should not be ‘read-in’ as limitations on any claims expressed more broadly.

### Definitions

The term “comprising” or derivatives thereof, eg “comprises”, if and when used in this document in relation to a combination of features or steps should not be taken to rule out the option of there being additional features or steps that have not been mentioned. The term is therefore inclusive, not exclusive.

### SUMMARY OF THE INVENTION

In this section references to the composition or features thereof mean any of the options given for the composition, unless it is made clear that only a particular option or options for the composition are being referred to.

According to one aspect of the invention there is provided a composition for topical treatment, comprising:
rapamycin as active ingredient;
vehicle, comprising:
   monolaurin, for example as glyceryl monolaurate; and
   monomyristin, for example as glyceryl monomyristate; and
water as a solvent.

Optionally the rapamycin is present in the amount of 0.5-5% wt.

Optionally the rapamycin is present in the amount of approximately 0.5% wt.

Optionally the rapamycin is present in the amount of approximately 1.0% wt.

Optionally the rapamycin is present in the amount of approximately 5.0% wt.

Optionally the composition is for treating angiofibromas.

Optionally the composition is for treating facial angiofibromas.

Optionally the composition is for treating cutaneous vascular lesions.

Optionally the composition is for treating port wine stains.

Optionally the composition is for treating port wine stains after laser treatment thereof.

### Monolaurin

Optionally the monolaurin comprises one or more of a monolaurate (eg glycerol monolaurate).

Optionally the monolaurin is present in the amount of approximately, 7-28% wt or approximately 5%-10% wt, and preferably approximately 7% wt.

### Monomyristin

Optionally the monomyristin comprises one or more of a monomyristate, eg glyceryl monomyristate.

Optionally the monomyristin, eg glyceryl monomyristate, is present in the amount of approximately 7%-28% wt or approximately 15%-25% wt, and preferably approximately 21% wt.

### Combination Quantities of Monolaurin and Monomyristin

Optionally the composition is such that it comprises:
a) 5%-9% wt Monolaurin 19%-23% wt Monomyristin;
b) 26%-30% wt Monolaurin 0%-2% wt Monomyristin;
c) 0%-2% wt Monolaurin 26-30% wt Monomyristin;
d) 12%-16% wt Monolaurin 12-16% wt Monomyristin;

Optionally the composition is such that it comprises:
a) about 7% wt Monolaurin about 21% wt Monomyristin;
b) about 28% wt Monolaurin about 0% wt Monomyristin;
c) about 0% wt about 28% wt Monomyristin;
d) about 14% wt Monolaurin about 14% wt Monomyristin;

For each of the combination quantities noted at each set of a)-d) above, the rapamycin is optionally present in an amount of about 1% wt.

### Some Preferences for the First Mentioned Aspect of the Invention

Preferably the composition comprises 0.5-5% wt rapamycin, 5%-9% wt Monolaurin and 19%-23% wt Monomyristin.

Preferably the composition comprises g 0.5-5% wt rapamycin, 7% wt Monolaurin and 21% wt Monomyristin.

Preferably the composition comprises 1% wt rapamycin, 7% wt Monolaurin and 21% wt Monomyristin.

Preferably the composition comprises 0.5-5% wt rapamycin, 12%-16% wt Monolaurin and 12%-16% wt Monomyristin.

Preferably the composition comprises 0.5-5% wt rapamycin, 14% Monolaurin and 14% wt Monomyristin.

Preferably the composition comprises 1% wt rapamycin, 14% Monolaurin and 14% wt Monomyristin.

Preferably the composition comprises 0.5-5% wt rapamycin, 17.5% (±2%) wt Monolaurin and 10.5% (±2%) wt Monomyristin.

Preferably the composition comprises 1% wt rapamycin, 17.5% wt Monolaurin and 10.5% wt Monomyristin.

Preferably the composition comprises 1% wt rapamycin, 21% wt Monolaurin and 7% wt Monomyristin.

Preferably the Monolaurin is glyceryl monolaurate and the Monomyristin is glyceryl monomyristate.

Preferably the composition formulated to be suitable for treating angiofibromas, cutaneous vascular lesions or port wine stains in humans.

In one preferred option the composition comprises:
a) 0.5-5% wt rapamycin;
b) vehicle comprising:
   7-28% wt monolaurin; and
   7-28% wt monomyristin; and
c) water as a solvent.

In another preferred option the composition comprises:
a) 0.5-5% wt rapamycin;
b) vehicle comprising:
   7-28% wt monolaurin; and
   7-28% wt monomyristin; and
c) water as a solvent.

In another preferred option the composition comprises:
a) 0.5-5% wt rapamycin;
b) vehicle comprising:
   10-18% wt monolaurin; and
   10-18% wt monomyristin; and
c) water as a solvent.

### Water Retainers

Optionally the composition comprises a water retainer compound, for example polyoxyethylene stearate, present in the amount of approximately 1% wt.

Optional other water retainers may be used, for example hyaluronic acid.

### Softeners

Optionally the composition comprises a softener, for example propylene glycol. Preferably the softener, e.g. propylene glycol, is present in the amount of approximately 2% wt.

Optional other softeners comprise one or more of petrolatum, lanolin, mineral oil, glycerin, lecithin and sorbitol

### Buffers

Optionally the composition comprises a buffer, for example citric acid anhydrous. Preferably the buffer, e.g., citric acid anhydrous, is present in the amount of approximately 2% wt. Preferably the buffer maintains pH of the composition in the range of 3-5, and more preferably in the range 3.5 to 4.5.

Optional other buffers comprise one or more of sodium bicarbonate and triethanolamine.

### Sequestrants

Optionally the composition comprises a sequestrant, for example disodium edetate. Preferably the sequestrant, e.g. disodium edetate, is present in the amount of approximately 0.05% wt.

Optional other sequestrants comprise one or more of citric acid and tetrasodium EDTA.

### pH Adjusters

Optionally the composition comprises hydroxide for pH adjustment, for example sodium hydroxide. Optionally the sodium hydroxide is present in the amount of approximately 0.18% wt.

### Preservatives

Optionally the composition comprises a preservative, for example potassium sorbate. Preferably the preservative, e.g. potassium sorbate, is present in the amount of approximately 0.2% wt.

Optional other preservatives for use comprise one or more of diazolidinyl urea, phenoxyethanol and sodium hydroxymethylglycinate.

### Water

Optionally the water is present in the amount of approximately 58%-72% wt.

Optionally the composition is as per any of the combinations mentioned above, formed such that the rapamycin is in the form of multiple layers of undissolved or suspended particles where each layer is substantially located between layers of the vehicle. Preferably the particles have a size in the range of 1-100 μm.

According to another aspect, the invention comprises the use of the components of one or more of the above statements, in the manufacture of a composition for topical treatment of angiofibromas.

According to a further aspect, the invention comprises a method of treating human angiofibromas with a composition according to one or more of the above statements.

According to a another aspect of the invention there is provided a method for treating one or more of the following conditions, comprising administration to a human a composition according to any of the statements above:
facial angiofibromas;
cutaneous vascular lesions; and
port wine stains (e.g. after laser treatment thereof).

Optionally the composition is administered to the human topically.

Optionally the composition is administered to the human so that it encounters their dermis.

Optionally the composition is self-administered by the person, or is administered to that person by another person.

Optionally the composition is contained in and is administered from a squeeze tube.

According to some embodiments of the invention the composition or method is stated as above except that it consists of, or essentially consists of, the features or steps stated in each case.

According to a further aspect, the invention comprises (or consists of, or consists essentially of) the use of—
rapamycin as active ingredient;
vehicle, comprising:
   monolaurin, for example as glyceryl monolaurate; and
   monomyristin, for example as glyceryl monomyristate; and
water as a solvent;

in the preparation of a composition as stated above for use in treating any of the conditions mentioned for the above method. Optionally the composition is in accordance with any of the options or preferences mentioned above.

### IMAGES

Some preferred embodiments of the invention are illustrated by reference of the following images, of which—

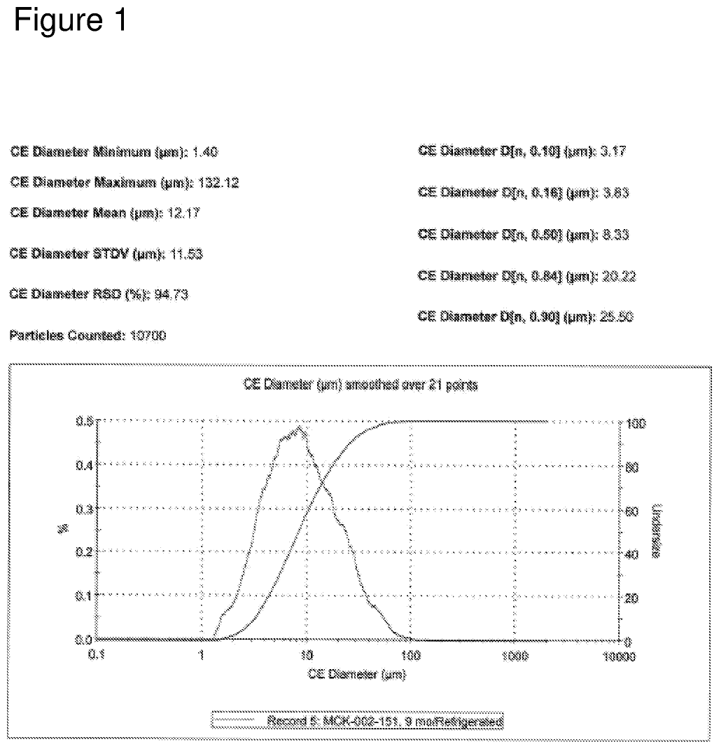

FIG. 1 shows the results obtained from particle size analysis of a 1% rapamycin composition for topical application; and

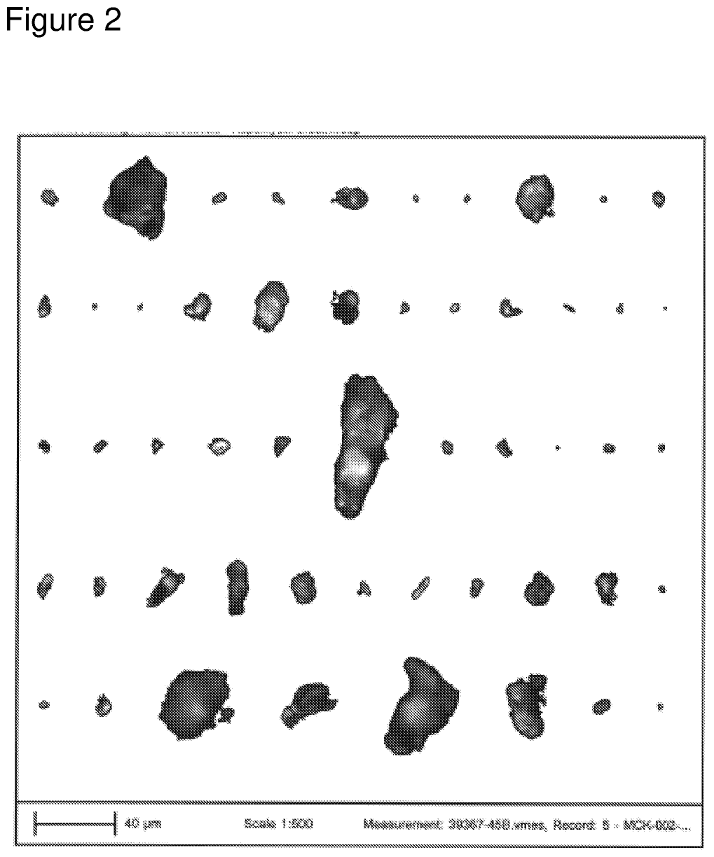

FIG. 2 shows images of rapamycin particles in the same 1% composition.

### DETAILED DESCRIPTION

### Example Formulations 1 & 2

Two preferred embodiments of the invention, namely Formulation 1 and Formulation 2, are for topical treatment of human angiofibromas, for example facial angiofibromas or cutaneous vascular lesions. The Formulations may be used for treating port wine stains, for example after laser treatment. They are composed substantially as follows—

| | | Amount (% wt) | |
|---|---|---|---|
| Component | Function | Formulation 1 | Formulation 2 |
| Rapamycin | Active ingredient | 0.50 | 1.00 |
| Glyceryl | Vehicle | 7.00 | 7.00 |
| monolaurate | | | |
| Glyceryl | Vehicle | 21.00 | 21.00 |
| Monomyristate | | | |
| Polyoxyethylene | Water retention | 1.00 | 1.00 |
| (100) stearate | | | |
| Propylene glycol | Softener | 2.00 | 2.00 |
| Citric acid | Buffer | 0.90 | 0.90 |
| anhydrous | | | |
| Disodium edetate | Sequestrant | 0.05 | 0.05 |
| Sodium hydroxide | pH adjustment | 0.18 | 0.18 |
| pallets | | | |
| Potassium sorbate | Preservative | 0.20 | 0.20 |
| Purified water | Solvent | 67.17 | 66.67 |

Compositions according to the invention, including Formulations 1 and 2, may be produced according to the following method.

### Production of a Water Phase Pre-Blend

A water phase pre-blend is prepared as follows:
the rapamycin and propylene glycol are combined and vortex mixed;
the water is added with continued vortex mixing;
the blend is then heated to 70° C. (±5° C.);
citric acid, disodium EDTA and potassium sorbate are added with vortex mixing until dissolved;
the temperate of the mixture is then regulated to 50° C.; and
the polyoxyethylene (100) stearate is added.

### Production of an Oil Phase Pre-Blend

An oil phase pre-blend is prepared as follows:
glyceryl monolaurate and glyceryl monomyristate are combined with slow speed vortex mixing and heating to 70° C. (±5° C.).

### Final Blend

The water and oil phase pre-blends, both at 70° C. (±5° C.), are combined and mixed thoroughly until homogenous. The mixture is then cooled slowly at a rate of 1° C. per minute and vortex mixed until it forms into a smooth, white and iridescent cream. When the cream had cooled to no more than 32° C. it is packaged in 30 mL aluminium tubes with polypropylene screw on caps.

### Example Formulations 3 & 4

Formulations 3 and 4 were produced for the same purpose and in the same way described for Formulations 1 and 2. They are as follows—

| | | Amount (% wt) | |
|---|---|---|---|
| Component | Function | Formulation 3 | Formulation 4 |
| Rapamycin | Active ingredient | 0.10 | 5.00 |
| Glyceryl | Vehicle | 7.00 | 7.00 |
| monolaurate | | | |
| Glyceryl | Vehicle | 21.00 | 21.00 |
| Monomyristate | | | |
| Polyoxyethylene | Water retention | 1.00 | 1.00 |
| (100) stearate | | | |
| Propylene glycol | Softener | 2.00 | 2.00 |
| Citric acid | Buffer | 0.90 | 0.90 |
| anhydrous | | | |
| Disodium edetate | Sequestrant | 0.05 | 0.05 |
| Sodium hydroxide | pH adjustment | 0.18 | 0.18 |
| pallets | | | |
| Potassium sorbate | Preservative | 0.20 | 0.20 |
| Purified water | Solvent | 72 | 66.1 |

### Stability Studies for Formulations 1-4

Studies were run to determine whether Formulations 1-4 are stable in storage at room temperature, namely 25° C. (±2° C.) and relative humidity of 60% (±5%). The results of the trial are as shown in the tables below. In each case T0 refers to test results at 0 months, T1 refers to test results at 1 month, T3 at 3 months, and so on. In each case references to a “specification” for test values relate to the patentee's preferred thresholds.

Formulation 1 (0.5% wt) at Room Temperature

| Parameter | T0 | T1 | T3 | T6 | T12 | T18 | T24 | T30 | T36 |
|---|---|---|---|---|---|---|---|---|---|
| Rapamycin | 106.4 | 109.4 | 105.3 | 98.1 | 97.6 | 105.4 | 95.8 | 91.6 | 91.2 |
| (% of the 0.5% label claim) | | | | | | | | | |
| (specification 90%-110%) | | | | | | | | | |
| Appearance | Smooth, | same | same | same | same | same | same | same | same |
| | white | | | | | | | | |
| | slightly | | | | | | | | |
| | iridescent | | | | | | | | |
| | cream, no | | | | | | | | |
| | phase | | | | | | | | |
| | separation | | | | | | | | |
| pH | 4.06 | 4.1 | 4.1 | 4.1 | 4.2 | 4.1 | 4.2 | 4.1 | 4.1 |
| (1 g sample in 10 g water) | | | | | | | | | |
| (specification 3.5-4.5) | | | | | | | | | |
| Water content % wt | 68.4 | 70.1 | NS | 67.2 | 69.0 | 69.9 | 62.3 | 65.2 | 65.7 |
| (specification 62.0-72.0) | | | | | | | | | |
| Total impurities % wt | 0.18 | 0.40 | 0.66 | 0.68 | 0.43 | 0.60 | 0.38 | 0.42 | 0.54 |
| (specification ≤2.5) | | | | | | | | | |
| Aerobic bacteria (cfu/g) | <10 | Not | ND | <10 | <10 | ND | <10 | ND | <10 |
| (specification <100) | | Done | | | | | | | |
| | | (ND) | | | | | | | |
| Yeast and mould (cfu/g) | <10 | ND | ND | <10 | <10 | ND | <10 | ND | <10 |
| (specification <10) | | | | | | | | | |

Footnotes:
ND = Not done. NS = Not sampled due to equipment malfunction. New Karl Fischer instrument [used to measure water] was subsequently installed at the laboratory.

Formulation 2 (1.0% wt) at Room Temperature

| Parameter | T0 | T1 | T3 | T6 | T12 | T18 | T24 | T30 | T36 |
|---|---|---|---|---|---|---|---|---|---|
| Rapamycin | 99.3 | 99.2 | 99.3 | 102.7 | 103.4 | 99.0 | 97.7 | 96.7 | 98.3 |
| (% of the 1.0% label claim) | | | | | | | | | |
| (specification 90%-110%) | | | | | | | | | |
| Appearance | Smooth, | same | same | same | same | same | same | same | same |
| | white slightly | | | | | | | | |
| | iridescent | | | | | | | | |
| | cream with | | | | | | | | |
| | no phase | | | | | | | | |
| | separation | | | | | | | | |
| pH | 4.10 | 4.1 | 4.1 | 4.1 | 4.2 | 4.1 | 4.2 | 4.1 | 4.1 |
| (1 g sample in 10 g water) | | | | | | | | | |
| (specification 3.5-4.5) | | | | | | | | | |
| Water content % wt | 71.5 | 62.9 | NS | 69.3 | 69.5 | 69.4 | 65.2 | 62.7 | 66.2 |
| (specification 62.0-72.0) | | | | | | | | | |
| Total impurities % wt | 0.19 | 0.39 | 0.47 | 0.32 | 0.37 | 0.40 | 0.36 | 0.35 | 0.5 |
| (specification ≤2.5) | | | | | | | | | |
| Aerobic bacteria (cfu/g) | <10 | ND | ND | <10 | <10 | ND | <10 | ND | <10 |
| (specification <100) | | | | | | | | | |
| Yeast and mould (cfu/g) | <10 | ND | ND | <10 | <10 | ND | <10 | ND | <10 |
| (specification <10) | | | | | | | | | |

Formulation 3 (0.1% wt) at Room Temperature

| Parameter | T0 | T1 | T3 | T6 | T12 | T18 |
|---|---|---|---|---|---|---|
| Rapamycin | 97.0 | 98.0 | 87.3 | 98.8 | 79.3 | 67.9 |
| (% of the 0.1% label claim) | | | | | | |
| (specification 90%-110%) | | | | | | |
| Appearance | Smooth, white | same | same | same | same | same |
| | slightly iridescent | | | | | |
| | cream with no | | | | | |
| | phase separation | | | | | |
| pH | 4.1 | 4.1 | 4.1 | 4.1 | 4.2 | 4.1 |
| (1 g sample in 10 g water) | | | | | | |
| (specification 3.5-4.5) | | | | | | |
| Water content % wt | 72.0 | NS | 63.6 | 64.7 | 65.8 | 63.8 |
| (specification 63.0-73.0) | | | | | | |
| Total impurities % wt | 0.25 | 0.55 | 0.38 | 0.52 | 0.56 | 0.40 |
| (specification ≤2.5) | | | | | | |
| Aerobic bacteria (cfu/g) | <10 | ND | ND | <10 | <10 | ND |
| (specification <100) | | | | | | |
| Yeast and mould (cfu/g) | <10 | ND | ND | <10 | <10 | ND |
| (specification <10) | | | | | | |

Formulation 4 (5.0% wt) at Room Temperature

| Parameter | T0 | T1 | T3 | T6 | T12 | T18 | T24 | T30 | T36 |
|---|---|---|---|---|---|---|---|---|---|
| Rapamycin | 102.1 | 103.8 | 108.1 | 103.5 | 108.9 | 105.6 | 104.5 | 106.4 | 107.3 |
| (% of the 5.0% label claim) | | | | | | | | | |
| (specification 90%-110%) | | | | | | | | | |
| Appearance | Smooth, white | same | same | same | same | same | same | same | same |
| | slightly | | | | | | | | |
| | iridescent | | | | | | | | |
| | cream with no | | | | | | | | |
| | phase | | | | | | | | |
| | separation | | | | | | | | |
| pH | 4.1 | 4.1 | 4.1 | 4.1 | 4.1 | 4.1 | 4.2 | 4.1 | 4.1 |
| (1 g sample in 10 g water) | | | | | | | | | |
| (specification 3.5-4.5) | | | | | | | | | |
| Water content % wt | 66.1 | 64.5 | NS | 65.9 | 63.2 | 65.5 | 58.1 | 58.9 | 61.6 |
| (specification 58.0-68.0) | | | | | | | | | |
| Total impurities % wt | 1.18 | 0.37 | 0.47 | 0.44 | 0.53 | 0.40 | 0.46 | 0.34 | 0.43 |
| (specification ≤2.5) | | | | | | | | | |
| Aerobic bacteria (cfu/g) | <10 | ND | ND | <10 | <10 | ND | <10 | ND | <10 |
| (specification <100) | | | | | | | | | |
| Yeast and mould (cfu/g) | <10 | ND | ND | <10 | <10 | ND | <10 | ND | <10 |
| (specification <10) | | | | | | | | | |

The trials showed that Formulations 1, 2 and 4 remained stable at room temperature. These results were remarkable, especially when it is considered that current market products lose stability significantly earlier. For example Nobelpharma has supplied the Japanese market with a 0.2% wt rapamycin gel formulation branded Rapalimus™. It has stability for only 12 months and only when refrigerated.

In further examples, formulations in the form of a 0.1% wt rapamycin cream were sourced and tested from the US market and also found to be unstable at room temperature, as follows—

| | Rapamycin (90- | | T1 | T3 | T6 |
|---|---|---|---|---|---|
| Source | 110%) | T0 | Month | month | month |

| | | | | |
|---|---|---|---|---|
| Restore Health | 91% | 42% | 15% | 8% |
| [Lot121005-021] | | | | |
| Doyles | 97% | 99% | 90% | 71% |
| Pharmacy [Uni | | | | |
| Texas] [LotDP1] | | | | |

Surprisingly Formulation 3 (0.1% Rapamycin) was found to be unstable. After 12 and 18 months storage the Rapamycin had degraded to only 79.3% and 67.9% of the label amount respectively. In comparison the higher strengths 0.5%-5% gave unexpectedly superior results. The better results for the higher strengths is difficult to explain and appears to be due to a surprising synergy.

### Example Formulations 5-11

The following further formulations were prepared in the same manner described above, for Formulations 1-4, and for the same therapeutic purpose—

| | | Amount (% wt) | | | | | | |
|---|---|---|---|---|---|---|---|---|
| | | Formulation No. | | | | | | |
| Component | Function | 5 | 6 | 7 | 8 | 9 | 10 | 11 |
| Rapamycin | Active ingredient | 1.0 | 1.0 | 1.0 | 1.0 | 1.0 | 1.0 | 1.0 |
| Monolaurin | Vehicle | 7.0 | 28.0 | 0.0 | 14.0 | 21.0 | 10.5 | 17.5 |
| Monomyristin | Vehicle | 21.0 | 0.0 | 28.0 | 14.0 | 7.0 | 17.5 | 10.5 |
| Polyoxyethylene | Water retention | 1.0 | 1.0 | 1.0 | 1.0 | 1.0 | 1.0 | 1.0 |
| (100) stearate | | | | | | | | |
| Propylene glycol | Softener | 2.0 | 2.0 | 2.0 | 2.0 | 2.0 | 2.0 | 2.0 |
| Citric acid anhydrous | Buffer | 0.9 | 0.9 | 0.9 | 0.9 | 0.9 | 0.9 | 0.9 |
| Disodium edetate | Sequestrant | 0.05 | 0.05 | 0.05 | 0.05 | 0.05 | 0.05 | 0.05 |
| Sodium hydroxide | pH adjustment | 0.18 | 0.18 | 0.18 | 0.18 | 0.18 | 0.18 | 0.18 |
| pallets | | | | | | | | |
| Potassium sorbate | Preservative | 0.20 | 0.20 | 0.20 | 0.20 | 0.20 | 0.20 | 0.20 |
| Purified water | Solvent | 66.67 | 66.67 | 66.67 | 66.67 | 66.67 | 66.67 | 66.67 |

These were subjected to storage stability tests at room temperature, being 25° C. (±2° C.) and relative humidity of 60% (±5%), over 6 months.

The results of the tests were as shown in the table below. In each case the T value indicates when the sample was tested, for example T0 indicates testing at 0 months, T1 at one month, and so on. The % figures show the proportion of the 1% wt label amount of rapamycin that remained at each test point. In cases where rapamycin is lower, this indicates that the compound has been the subject of a degradation reaction.

Stability Tests on Formulations 5-11

| | T0 | T1 | T3 | T6 | Change | |
|---|---|---|---|---|---|---|
| Formulation | % | % | % | % | % | Assessment |
| 5 | 98 | 108.8 | 113 | 105.1 | +7.1 | Stable |
| 6 | 101.2 | 108.1 | 95.5 | 95 | −6.2 | Stable |
| 7 | 91.2 | 102.5 | 94.5 | 103.7 | +12.5 | Stable |
| 8 | 98.4 | 98.4 | 99.6 | 101.4 | +3.0 | Stable |
| 9 | 95.5 | 95.5 | 106.7 | 83.1 | −12.4 | Unstable |
| 10 | 118 | 118 | 105.1 | 100.9 | −17.1 | Unstable |
| 11 | 107.6 | 107.6 | 108.3 | 97.6 | −10.0 | Marginally |
| | | | | | | stable |

Formulations 5-11 were also retested at each time period to see whether they had developed excess water content. The samples were considered to pass if the met limits of between 62-72% wt. The results were as follows—

Water Content Tests

| Formu- | | | | | |
|---|---|---|---|---|---|
| lation | Result [62-72%] | T0 | T1 | T3 | T6 |
| 5 | Passed at all of T0-T6 | 66% | 65.9% | 66% | 70.9% |
| 6* | Failed at T1 & T6 | 66.7% | 61.2% | 70.1% | 76.5% |
| 7 | Failed at T0 | 75.7% | 63.3% | 64.8% | 68.2% |
| 8 | Failed at T3 | 69.6% | 64.9% | 59.7% | 65.9% |
| 9 | Failed at T1 | 66.4% | 59.9% | 67.7% | 68.2% |
| 10 | Failed at T1 | 72% | 58.8% | 67.5% | 66.4% |
| 11 | Failed at T1 | 67.4% | 58.1% | 66.4% | 66.0% |

Footnote
*Additionally Formulation 6 failed the appearance specification

| Specification | T0 | T1 | T3 | T6 |
|---|---|---|---|---|
| Smooth white, | Does not | Conforms | Slightly phase | Does not |
| slightly iridescent | conform - | | separation | conform - |
| cream with | Very runny, | | (Slightly | slightly |
| visually no phase | mild phase | | curdled) | curdled |
| separation | separation | | | |

For a formulation to be useful the rapamycin content should not fall below 90% of the original amount as this would represent a 10% loss in potency of the active ingredient and would be consistent with the formulation not being stable due to hydrolysis/oxidation of the active ingredient.

In terms of water content, each composition is an oil-in-water product and so the amount of water present is relevant to maintaining a stable homogenous formulation. Water content outside accepted limits is indicative of a lack or loss of formulation stability. Skin absorption was measured using a Franz cell diffusion apparatus. Studies for the preferred Formulations 1 & 2 showed absorption in a human skin model and penetrability profiles higher than for Formulation 3, with a validated mass-balance recovery. More specifically, absorption through healthy human skin into 5% bovine serum albumin, 0.9% NaCl in water was measured using the Franz cell. Only the higher strength formulations, Formulations 1 & 2, met the acceptance criteria for absorption and penetration across the human skin. Formulation 3 failed this test. This failure for Formulation 3 was unrelated to stability as the tests were undertaken with freshly made materials. The difference in skin absorption and penetration was surprising.

FIGS. 1 and 2 illustrate characteristics of the rapamycin active ingredient for the 1% cream identified above as Formulation 2. The information was obtained by analysing the Formulation using a Malvern Morphologi GS3 image analyser. This is effectively an automated microscope that scanned the Formulation to detect 3D shape features of the rapamycin particles.

Referring to FIG. 1, the CE Diameter (‘circle equivalent diameter’) values represent particle size on a number weighted basis. In other words it represents the particles as 2-dimensional shapes converted to the nearest applicable circle, for which the diameter is then calculated. Referring to the particular notation shown, D[n,0.10](μm): 3.17 means that 10% of the particles are 3.17 μm or smaller on a number of particles basis; D[n,0.16] (μm): means that 16% are smaller than 3.83 μm; and D[n,0.50] (μm): means that 50% are smaller than 8.33 μm on a number of particle basis, etc

Referring to FIG. 2, the images are representative of the shape and relative size of rapamycin particles in Formulation 2.

FIGS. 1 and 2 illustrate that the rapamycin is not dissolved in the rest of the composition but rather sits suspended in the carrier or vehicle component.

In terms of disclosure, this document hereby discloses each item, feature or step mentioned herein in combination with one or more of any of the other item, feature or step disclosed herein, in each case regardless of whether such combination is claimed.

While some preferred embodiments of the invention have been described by way of example, it should be understood that modifications and improvements can occur without departing from the scope of the following claims.

1. A composition for topical treatment, comprising:
a) 0.5-5% wt rapamycin as active ingredient;
b) vehicle, comprising:
7-28% wt monolaurin; and
7-28% wt monomyristin; and
c) water.

2. (canceled)

3. A composition according to claim 1, wherein the rapamycin is present in the amount of approximately 0.5% wt.

4. A composition according to claim 1, wherein the rapamycin is present in the amount of approximately 1.0% wt.

5. A composition according to claim 1, wherein the rapamycin is present in the amount of approximately 5.0% wt.

6.-10. (canceled)

11. (canceled)

12. A composition according to claim 1, wherein the monolaurin is present in the amount of approximately 5%-10% wt.

13.

14. A composition according to claim 1, wherein the monomyristin, is present in the amount of approximately 15%-25% wt.

15. A composition according to claim 1, comprising:
a) 5%-9% wt Monolaurin+19%-23% wt Monomyristin;
b) 12%-16% wt Monolaurin+12-16% wt Monomyristin.
c) about 7% wt Monolaurin+about 21% wt Monomyristin; or
d) about 14% wt Monolaurin+about 14% wt Monomyristin.

16. (canceled)

17. (canceled)

18. A composition according claim 1, comprising a water retainer.

19. A composition according to claim 18, wherein the water retainer comprises polyoxyethylene stearate.

20. A composition according to claim 1, comprising a softener.

21. A composition according to claim 20, wherein the softener comprises one or more of propylene glycol, petrolatum, lanolin, mineral oil, glycerin, lecithin and sorbitol.

22. A composition according claim 1, comprising a buffer.

23. A composition according to claim 22, wherein the buffer comprises one or more of citric acid anhydrous, sodium bicarbonate and triethanolamine.

24. A composition according to claim 1, comprising a sequestrant.

25. A composition according to claim 24, wherein the sequestrant comprises one or more of disodium edetate, citric acid and tetrasodium EDTA.

26. A composition according to claim 1, comprising hydroxide for pH adjustment.

27. A composition according to claim 1, comprising preservative.

28. A composition according to claim 27, wherein the preservative comprises one or more of potassium sorbate, diazolidinyl urea, phenoxyethanol and sodium hydroxymethylglycinate.

29. A composition according to claim 1, wherein the water is present in the amount of 58%-72% wt.

30. (canceled)

31. A composition according to claim 1, comprising: (a) 0.5-5% wt rapamycin, 5%-9% wt Monolaurin and 19%-23% wt Monomyristin; (b) 0.5-5% wt rapamycin, 7% wt Monolaurin and 21% wt Monomyristin; (c) 1% wt rapamycin, 7% wt Monolaurin and 21% wt Monomyristin; (d) 0.5-5% wt rapamycin, 12%-16% wt Monolaurin and 12%-16% wt Monomyristin; or 0.5-5% wt rapamycin, 14% wt Monolaurin and 14% wt Monomyristin.

32.-44.

45. A method for treating a condition selected from the group consisting of: angiofibromas; cutaneous vascular lesions; and port wine stains, the method comprising topically administering a composition according to claim 1.
